(19) United States
(12) Patent Application Publication
(10) Pub. No.: US 2005/0048568 A1
(43) Pub. Date: Mar. 3, 2005

(54) High sensitivity protein workstation and techniques

(76) Inventors: Thomas Laurell, Lund (SE); Johan Nilsson, Bjarred (SE); Gyorgy Marko-Varga, Malmo (SE)

Correspondence Address: LADAS & PARRY LLP, 224 SOUTH MICHIGAN AVENUE, SUITE 1200, CHICAGO, IL, 60604, US

(21) Appl. No.: 10/498,072

(22) PCT Filed: Dec. 11, 2002
(86) PCT No.: PCT/SE02/02286

(30) Foreign Application Priority Data
Dec. 11, 2001 (SE) 0104125-0

(51) Int. Cl. G01N 33/53; C12M 1/34

(52) U.S. Cl. 435/7.1; 435/287.2

(57) ABSTRACT

A method of selecting and identifying bio-molecules present in a bio-sample is disclosed. The method comprises the steps of: obtaining a bio-sample; amplifying (2) the bio-molecules present in the bio-sample to improve the case of detection of said bio-molecules; separating the bio-molecules in said amplified bio-sample; depositing (3) the amplified bio-molecules on to a suitable media. Detecting means are then used to identify or detect the presence of bio-molecules in said amplified and separated sample wherein said amplification step occurs in close physical proximity to said deposition step. A device (1) for carrying out the method is also disclosed as is a protein chip library produced by the device or method.

This invention relates to a protein workstation and methods of detecting proteins. It further relates to libraries of proteins located by the said workstation and/or methods.

The workstation provides methods and apparatus for the selection and identification of bio molecules present in a biological sample. Separating bio molecules present in a complex mixture generates a two-dimensional array. The identity and relative abundance of bio molecules detected by known methods of imaging the two dimensional array.

The workstation provides a high sensitivity protemics platform based on monocrystalline silicon micro-fabricated technology.

The output of the station allows automatic identification of target proteins and target protein complexes from biological samples. Techniques including an interface to high-sensitivity mass spectrometry can also be utilised, as are methods of automatically searching databases using search routines for protein identification.

### BACKGROUND TO THE INVENTION

Methods exist to select and identify bio molecules present in a complex biological sample. However such methods are somewhat slow and labour intensive

Such methods do not make the best use of the bio-sample as they use large amounts of material when deposition is made on sample plates. This also reduces the number of sample deposits that can be made in a given area.

The present invention provides a workstation and a method of selecting bio-molecules in which the detection ability is enhanced and a greater number of sample deposits in a given area can be provided.

### DESCRIPTION OF THE PRESENT INVENTION

One form of he present invention comprises a method of selecting and identifying bio-molecules present in a bio-sample comprising the steps of: obtaining a bio-sample; amplifying the bio-molecules present in the bio-sample to improve the ease of detection of said bio-molecules; separating the bio-molecules in said amplified bio-sample; separating the amplified bio-molecules in terms of molecular weight; using identification or detecting means to identify or detect the presence of bio-molecules in said amplified and separated sample.

The amplified and separated bio sample can store afterwards under appropriate conditions.

Another aspect of the present invention provides a protein chip library produced by the method of; obtaining a bio-sample; amplifying the bio-molecules present in the bio-sample to improve the ease of detection of said bio-molecules; separating the bio-molecules in said amplified bio-sample; separating the amplified bio-molecules in terms of molecular weight; using identification or detecting means to identify or detect the presence of bio-molecules in said amplified and separated sample.

In known devices there is a considerable amount of “dead space” that is, volume between the extraction or amplification area and the deposition portion. This means that extra volume of bio-sample is required and that there is a delay in the deposition. The present invention overcomes the abovementioned disadvantage by reducing the dead space resulting in more efficient and flexible apparatus.

### DESCRIPTION OF THE FIGURES

FIG. 1 shows a cross sectional view of a device according to a preferred form of the present invention, said device employing a piezoelectric element to deposit or dispense the bio-sample;

FIG. 2 shows a plan view of the device as illustrated in FIG. 1;

FIG. 3 shows a particularly preferred form of the present invention in which the bio-sample amplification occurs in a volume, which is separable from the remainder of the device thus allowing rapid replacement;

FIG. 4 shows a form of the present invention in which the amplification of the bio-sample occurs in a volume which is partitioned by means of a weir (6); and

FIG. 5 shows an alternative form of the invention illustrated in FIG. 3 wherein the bio-sample volume is portioned by means of a series of parallel upright members (7).

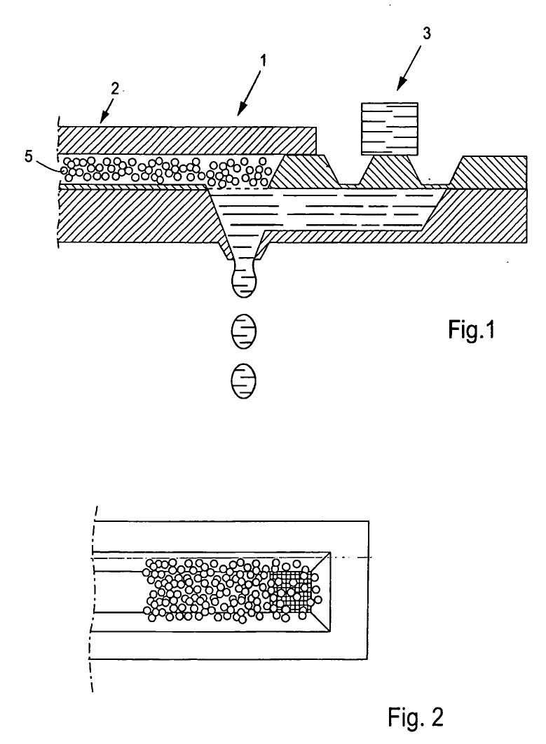

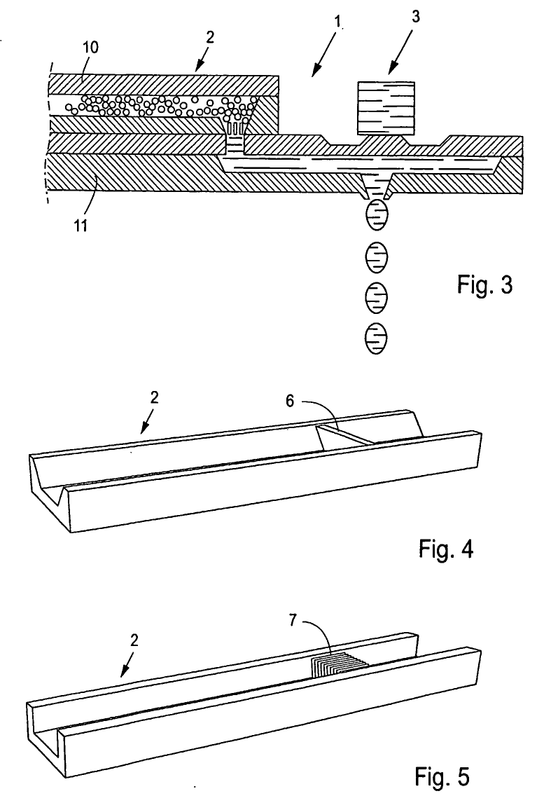

The step of micro-extraction or bio-sample amplification can be performed by utilising a number of known technique. The use of these will know be described along with situations when they will be selected for use.

The micro-extraction volume or channel (2) in which the amplification step occurs may be filled with packed beads (5) this technology is well known in the field to which the invention relates. There have been developed a number of theoretical models, which allow calculations to be made so that exact fluidic flow profiles can be determined. In addition simulations can be made to calculate exact peak capacities, binding kinetics and displacement volumes, these are all useful in ensuring protein amplification efficiency.

Another technique is the use of a porous membrane (not illustrated), this has the advantage of providing a low back pressure, which allows separation to be made based upon the size and shape of bio-macromolecules thereby isolating specific fractions of the sample.

The channel can be provided with porous channel walls this technique allows the use of high linear flow rates, thereby providing high-speed analyte processing with low backpressures. The use of parallel flow configurations will allow simultaneous multi-sample handling; one embodiment of the present invention is as herein described.

Yet another technique that can be used in preferred forms of the present invention is the use of micro and nano sized monolitic structures, that is, very well defined pores in the structure having a narrow pore size window. High capacities can be obtained on small volumes and sizes; this can be useful in the recoveries of proteins present in bio-samples.

Yet another technique that may be utilised in preferred forms of the present invention is the use of micro porous polymeric structures, that is, specific polymer networks with defined pore sizes that can be synthesized in-situ which will allows highly controlled separations of proteins to be made. Capacities can be made high, as can the linear flow rate through this packed structure thereby allowing high throughput operations in forms of the invention requiring this.

Each of the aforementioned techniques can be performed by; pressure driven or electrically driven devices or other suitable techniques.

Chromatographic separation where we will utilise mechanisms of
Chemical Binding
   i/ size exclusion—in samples where fractionation is required based upon size.
   ii/ hydrophobic interactions—utilisation of reversed phase separation mechanisms whereby peptides and proteins will be separated by its hydrophobicity.
   iii/ polar interactions—silanol, and other types of polar functionalities readily interact with polar peptides/proteins and can be separated based upon polar chromatographic interactions.
Affinity Binding by
   i/ Chiral affinity—chiral small molecules may lend itself to be used as selective ligands for proteins/peptides to interact with whereby separations will be obtained.
   ii/ Metal affinity—Chelation by metal ion interaction of amine, and or carboxy-hydroxy functional groups, as well as Nickel ion-Histidine peptide residues, iron-, Gallium-ions and phosphate functionalities on peptides binds strongly.
Biochemical Bindings:
   iii/ Antibody binding—Traditional biochemical bindings antibody-antigen immunoaffinity bindings with both weak-medium-strong affinities with binding constants ranging 10⁷-10⁹
   iv/ biotin avidin—affinity reagents utilising either Avidin or Biotin bound to peptides and either Avidin or Biotin on a solid support will selectively isolate peptides from complex sample mixtures due to the high affinity between Avidin/Biotin.

The present invention uses very small sample sizes and therefore sample losses due to for example adsorption by exposed surfaces is minimised. The downscaling in size of the target plate also allows many more sample positions to be generated in a given area. For example it has been found that 1000 to 3000 sample positions can be generated in an area in which the previous technique of laser desorption allowed only 100-400. This s illustrated in the FIG. 4. Such an increase in number of samples available in a given are has obvious advantages particularly in relation to the storage of target plates in order to form a protein or protein complex library.

Libraries of such proteins identified using the workstation and methods herein described can be produced and used for future reference. Such libraries may prove useful in future research and they provide an easy method of identification of the biological effect of molecules. The library may consist of a series of protein chips, which are stored under the appropriate conditions to ensure that they are not degraded over time.

Various means of depositing (3) the material are known in the art to which the invention relates for example: peizo-electric dispensing, magnetostrictive actuated dispensing or bubble-jet dispensing. These techniques have advantages and disadvantages resulting from the means by which dispensing occurs. For example bubble-jet dispensing may cause heating of the substance dispensed and therefore prove unsuitable for use with heat sensitive samples.

Finally, it will be understood by a person skilled in the art that the present invention has been described in at least one preferred embodiment and can be modified in many different ways without departing from the scope of the invention as defined in the appended claims.

1. A method of selecting and identifying bio-molecules present in a bio-sample comprising the steps of:
obtaining a bio-sample;
amplifying the bio-molecules present in the bio-sample to improve the ease of detection of said bio-molecules;
separating the bio-molecules in said amplified bio-sample;
depositing the amplified bio-molecules on to a suitable media;
using identification or detecting means to identify or detect the present of bio-molecules in said amplified and separated sample wherein said amplification step occurs in close physical proximity to said deposition step, and where said close physical proximity corresponds to a distance less than 5 millimeters.

2. A method of selecting and identifying bio-molecules present in a bio-sample as claimed in claim 1 wherein said amplification step and said deposition step take place so that the volume of bio-sample between said steps is less than 0.25 micro-litres.

3. A method of selecting and identifying bio-molecules present in a bio-sample as claimed in claim 1 wherein the deposition step is a non-contact one.

4. A method of selecting and identifying bio-molecules present in a bio-sample as claimed in claim 1 wherein the method is conducted in a flow-through device.

5. A method of selecting and identifying bio-molecules present in a bio-sample as claimed in claim 1 wherein the device for carrying out said method is a one piece device.

6. A method of selecting and identifying bio-molecules present in a bio-sample as claimed in claim 1 wherein the device for carrying out said method utilizes a portion for performing said amplification step and a separate portion for carrying out said deposition step said portions being sandwiched together.

7. A method of selecting and identifying bio-molecules present in a bio-sample as claimed in claim 1 wherein the amplified sample in deposited on a silicon plate by means of one of piezo-electric dispensing, magnetostrictive actuated dispensing or bubble-jet dispensing.

8. A method of selecting and identifying bio-molecules present in a bio-sample as claimed in claim 1 wherein the amplified and separated bio sample is stored afterwards.

9. A device for selecting and identifying bio-molecules present in a bio-sample comprising:
means for obtaining a bio-sample;
means for amplifying the bio-molecules present in the bio-sample to improve the ease of detection of said bio-molecules;
means for separating the bio-molecules in said amplified bio-sample;
means for depositing the amplified bio-molecules on to a suitable media;
identification or detecting means to identify or detect the presence of bio-molecules in said amplified and separated sample wherein said amplification means comprises micro-extraction or bio-sample amplification means comprising a channel or volume provided with either packed beads, a porous membrane, porous channel walls, micro-sized monolithic structures, nano-sized monolithic structures or micro porous polymeric structures, or a combination thereof; and
wherein said depositing means comprises either a piezo-electric dispenser or a magnetostrictive actuated dispenser of a bubble-jet dispenser and wherein said amplification means and said depositing means are arranged in close physical proximity to each other and where said close physical proximity corresponds to a distance less than 5 millimeters.

10. A device for selecting and identifying bio-molecules present in a bio-sample as claimed in claim 9 wherein said amplification means and said depositing means are located so that the volume of bio-sample between said means is less than 0.25 micro-litres.

11. A device for selecting and identifying bio-molecules present in a bio-sample as claimed in claim 9 wherein the device is a one piece device.

12. A device for selecting and identifying bio-molecules present in a bio-sample as claimed in claim 9 wherein the amplification means and the depositing means are sandwiched together.

13. A device for selecting and identifying bio-molecules present in a bio-sample as claimed in claim 9 wherein the amplified sample is deposited on a silicon plate by means of piezo-electric deposition.

14. A device for selecting and identifying bio-molecules present in a bio-sample as claimed in claim 9 wherein the device is a flow-through one.

15. A device for selecting and identifying bio-molecules present in a bio-sample as claimed in claim 9 wherein the amplified and separated bio sample is stored afterwards.

16. A protein chip library produced by aid of the method of claim 1.

17. A method for preparing total protein compositions from thin sections of frozen tissue, cells and biological specimens, includes both cryostat sections and films of biomaterials as claimed in claim 1.
